(19) United States
(12) Patent Application Publication
(10) Pub. No.: US 2013/0018280 A1
(43) Pub. Date: Jan. 17, 2013

(54) GUIDE WIRE

(71) Applicants: Yutaka TANO, Fujinomiya-shi (JP); Tomokane KUROSAWA, Fujinomiya-shi (JP)

(72) Inventors: Yutaka TANO, Fujinomiya-shi (JP); Tomokane KUROSAWA, Fujinomiya-shi (JP)

(73) Assignee: TERUMO KABUSHIKI KAISHA, Shibuya-ku (JP)

(21) Appl. No.: 13/565,495

(22) Filed: Aug. 2, 2012

Related U.S. Application Data
(63) Continuation of application No. PCT/JP2011/052410, filed on Feb. 4, 2011.

(30) Foreign Application Priority Data
Feb. 5, 2010 (JP) 2010-023942

(51) Int. Cl. A61M 25/09

(52) U.S. Cl. 600/585

(57) ABSTRACT

A guide wire includes a core wire which forms a line shape formed from a flexible metal material. A distal portion of the core wire is arranged in an axial line direction of the core wire and includes a plurality of deformable portions, which are easily-deformable in a specific direction on a plane (x-y plane) with the axial line of the core wire being a normal line, and easily-deformable directions of the adjacent easily-deformable portions among the respective easily-deformable portions are different from each other.

### CROSS REFERENCES TO RELATED APPLICATONS

This application is a continuation of International Application No. PCT/JP2011/052410 filed on Feb. 4, 2011 and claims priority to Japanese Patent Application JP1020-023942 filed in the Japanese Patent Office on Feb. 5, 2010, the entire content of both of which is incorporated herein by reference.

### TECHNICAL FIELD

The present invention generally pertains to a guide wire.

### BACKGROUND DISCUSSION

When a catheter is inserted into a body lumen such as a digestive tract, a blood vessel or the like, a guide wire is used to guide the catheter to a target region of the body lumen. The guide wire passes through the inside of the catheter. In addition, observation and/or treatment of a body lumen or the like is carried out using an endoscope. The guide wire guides the catheter, which has been inserted into the endoscope or a lumen of the endoscope to a target region of a body lumen or the like. An example of such a guide wire is disclosed in United States Application Publication No. 2008/0306468 (hereinafter “US 2008/0306468”).

The guide wire described in US 2008/0306468 includes a main body portion and a distal portion which is provided on the distal side of the main body portion and which extends toward a direction inclined to the axial line of the main body portion. The distal portion has a first bend portion, a second bend portion which bends in the opposite direction to the first bend portion, and a third bend portion which bends in the opposite direction to the second bend portion and are continuously provided in sequence from the main body portion side. The distal portion is comprised of an elongated member having a flat-plate shape, which is bendable toward the surface direction thereof.

In such a guide wire as described in US 2008/0306468, if the distal most end thereof gets into a lateral branch, the flexible distal portion bends and advances into the main blood vessel with the bent portion being a leading head, the erroneous intrusion of the guide wire into a lateral branch can be prevented.

However, for the guide wire as described in US 2008/0306468, since the distal portion of the elongated member forms a flat-plate shape, the bending thereof toward the surface direction is easy but conversely, bending thereof toward a direction other than that direction (in particular, toward a direction perpendicular to that surface direction) is difficult. Thus, the distal portion is not easily bendable toward other directions. Therefore, depending on the position of the lateral branch, on the extending direction or the like, the distal portion does not bend according to the operator's wish, and it is not possible to prevent its erroneous intrusion into a lateral branch.

### SUMMARY

According to one aspect, a guide wire is disclosed whose distal portion is easily bendable toward whichever direction and which is capable of preventing or suppressing erroneous intrusion thereof into a lateral branch.

According to another aspect, a guide wire, which includes an elongated core wire having a main body portion and a distal portion positioned on a distal side of the main body portion. The distal portion has a plurality of adjacent plate-shaped members, each of the plurality of adjacent plate-shaped members having a surface direction on a plane of an axial line of the elongated core wire, and wherein the surface directions of the adjacent plate-shaped members among the plurality of adjacent plate-shaped members are different from each other.

According to a further aspect, a guide wire includes an elongated core wire having a main body portion and a distal portion positioned on a distal side of the main body portion. The distal portion has a plurality of notch portions, each of the plurality of notch portions formed in pairs, which face each other through a central axis of the core wire. The plurality of notch portions are located at even intervals in an axial direction of the core wire and wherein each adjacent notch portion among the plurality of notch portions deviate from each other in a circumferential direction of the core wire.

According to another aspect, a guide wire includes a core wire formed from a flexible metal material. A distal portion of the core wire is arranged in an axial line direction of the core wire and includes a plurality of deformable portions, each of the plurality of deformable portions having a surface direction on a plane with the axial line of the core wire. The surface directions of adjacent deformable portions among the respective deformable portions are different from each other.

According to a further aspect, a guide wire disclosed herein includes a core wire which forms a line shape and which is made of metal material having flexibility, and wherein the distal portion of the core wire is arranged in the axis direction of the core wire and includes a plurality of easily-deformable portions, which are easily-deformable in a specific direction on a plane with the axial line of the core wire being a normal line, and easily-deformable directions of the adjacent easily-deformable portions among the respective easily-deformable portions are different from each other.

According to another aspect, each of the easily-deformable portions of the guide wire form a rotationally asymmetric shape with respect to the axial line of the core wire.

According to a further aspect, each of the easily-deformable portions of the guide wire form a plate-piece shape, and each surface direction of the adjacent easily-deformable portions among the respective easily-deformable portions are different from each other.

According to another aspect, the adjacent easily-deformable portions among the respective easily-deformable portions of the guide wire are provided in an interlocked manner.

According to a further aspect, the distal portion of the core wire of the guide wire includes a plurality of notch portions, which are arranged in the axial direction of the core wire, and the adjacent notch portions among the respective notch portions deviate from each other in the circumferential direction of the core wire, and regions of the core wire, in which the notch portions are formed, constitute the easily-deformable portions.

According to another aspect, each notch portion of the guide wire is formed in pairs so as to face each other through the central axis of the core wire.

According to a further aspect, the directions in which the respective easily-deformable portions are easily-deformable deviate continuously in one circumferential direction of the core wire in sequence from the easily-deformable portion positioned on the distal side of the core wire toward the easily-deformable portion positioned on the proximal side thereof.

According to another aspect, the directions in which the respective easily-deformable portions are easily-deformable deviate at even intervals in sequence from the easily-deformable portion positioned on the distal side of the core wire toward the easily-deformable portion positioned on the proximal side thereof.

According to a further aspect, the direction in which the easily-deformable portion positioned on the most distal side of the core wire among the plurality of easily-deformable portions is easily-deformable and the direction in which the easily-deformable portion positioned on the most proximal side thereof is easily-deformable deviate from each other by 90° or more in the one circumferential direction.

According to another aspect, the easily-deformable portion positioned on the most distal side of the core wire is among the plurality of easily-deformable portions to constitute the distal end of the core wire.

According to a further aspect, at least the distal portion of the core wire is covered by a coating layer.

### BRIEF DESCRIPTION OF DRAWINGS

FIG. 1 is a perspective view showing an embodiment of a guide wire.

FIG. 2 is a plan view of a distal portion of a core wire of the guide wire as shown in FIG. 1.

FIG. 3 is a perspective view showing a modified configuration of the distal portion of the core wire of the guide wire as shown in FIG. 1.

FIG. 4 is a perspective view showing a modified configuration of the distal portion of the core wire of the guide wire as shown in FIG. 1.

FIG. 5 is a perspective view showing a modified configuration of the distal portion of the core wire of the guide wire as shown in FIG. 1.

FIGS. 6A to 6C are explanatory views schematically showing how the guide wire as shown in FIG. 1 advances inside a blood vessel.

FIG. 7 is a perspective view showing another embodiment of a guide wire.

FIG. 8 is a cross-sectional view taken along the A-A line in FIG. 7.

FIG. 9 is a perspective view showing a further embodiment of a guide wire.

FIG. 10 is a perspective view showing another embodiment of a guide wire.

FIG. 11 is an exploded view of the guide wire as shown in FIG. 10.

### DETAILED DESCRIPTION

Set forth below is a detailed description of examples of embodiments of the guide wire disclosed here. The detailed description which follows describes features and aspects of the guide wire with reference to the attached drawings.

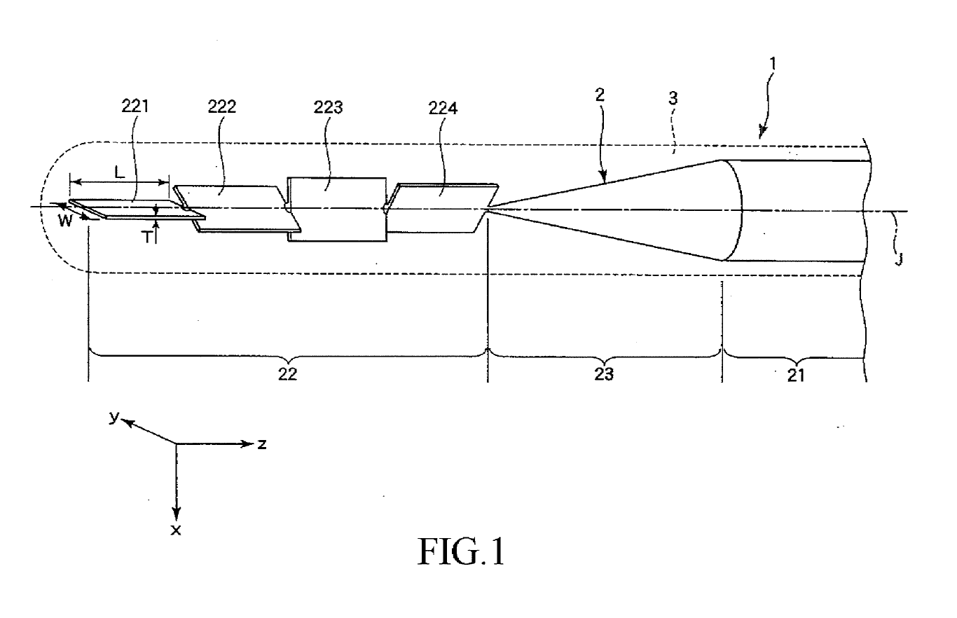

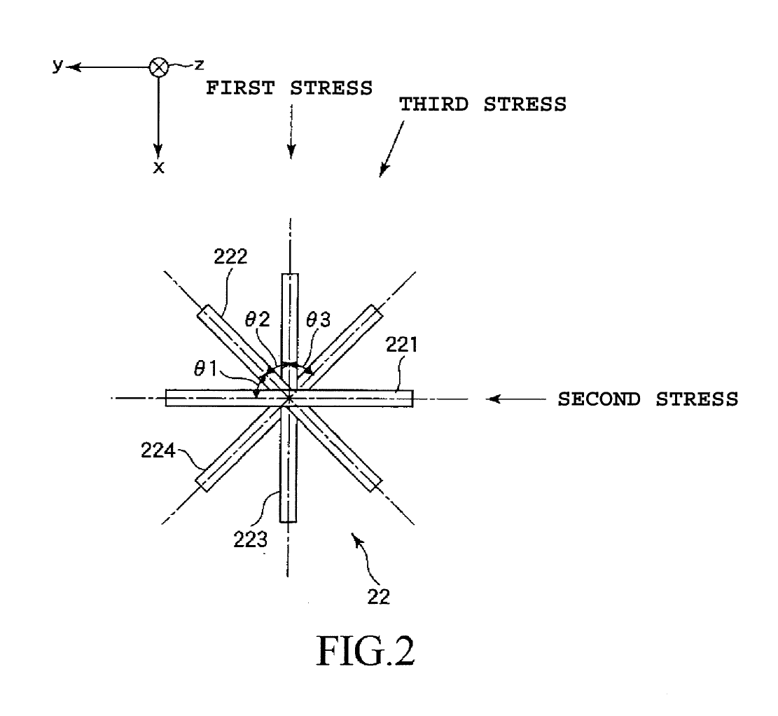

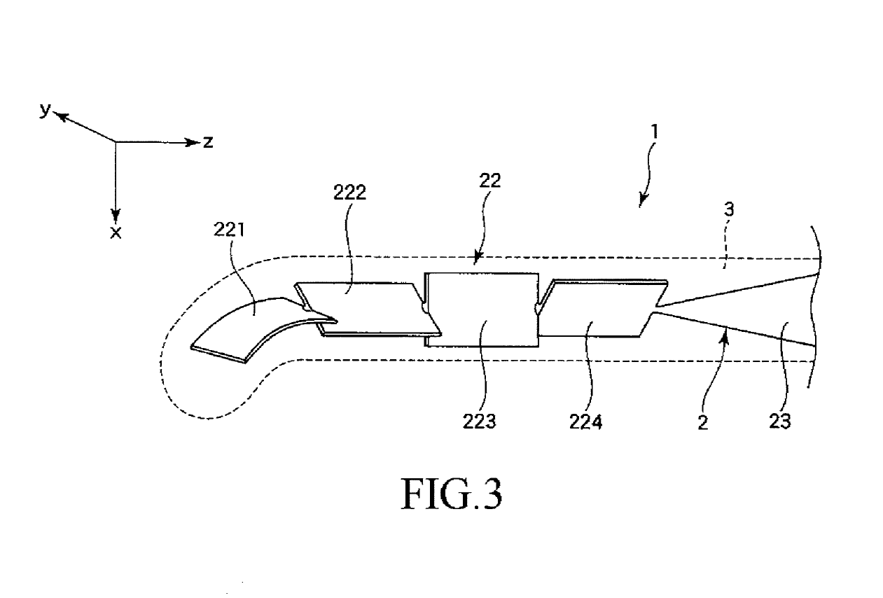

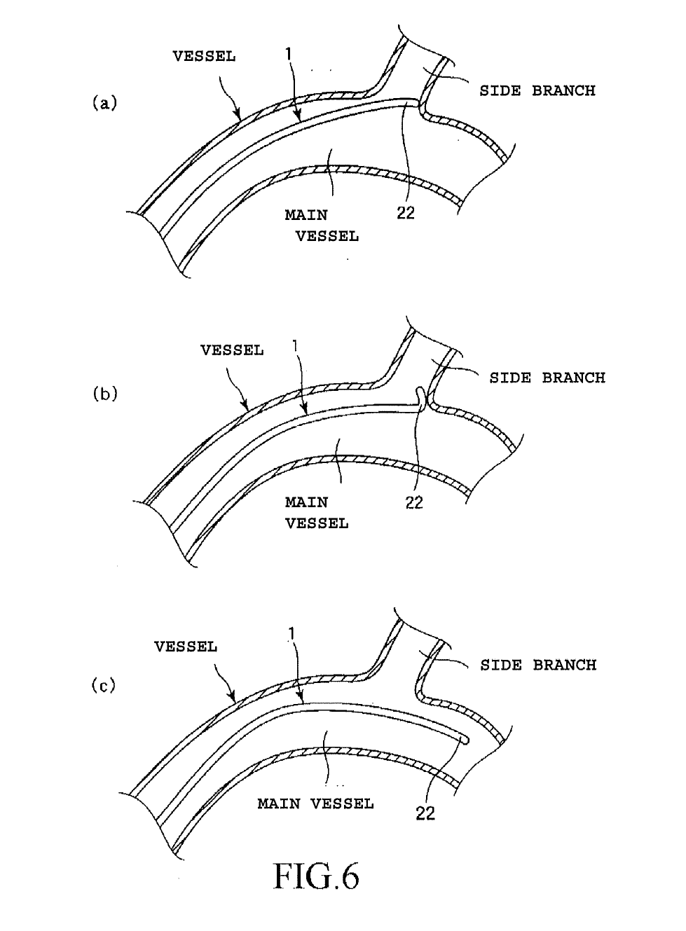

FIG. 1 is a perspective view showing an embodiment of a guide wire. FIG. 2 is a plan view of a distal portion of a core wire of the guide wire as shown in FIG. 1. FIG. 3 to FIG. 5 are perspective views showing modified configurations of the distal portion of the core wire of the guide wire as shown in FIG. 1. FIGS. 6A to 6C are explanatory views schematically showing how the guide wire as shown in FIG. 1 advances inside a blood vessel. For the sake of convenience in the following description, the right side in FIG. 1 (similarly also in FIGS. 2 to 5) will be referred to as “proximal end” and the left side will be referred to as “distal end”. In addition, in FIG. 1 (similarly also in FIGS. 2 to 5), in order to facilitate understanding, the thickness direction of the guide wire is illustrated schematically in an exaggerated form, so that the ratio between the length direction and the thickness direction is different from the actual ratio and is not necessarily intended to be an accurate illustration of the relative dimensions of part and features of the guide wire. In addition, as shown in FIG. 1 (similarly also in FIGS. 2 to 5), three axes perpendicular to one other are denoted by x-axis, y-axis and z-axis respectively, and the z-axis among them is set to be parallel with the axial line direction of the core wire.

A guide wire 1 shown in FIG. 1 is a guide wire for catheter or trans-endoscope, which is used by being inserted into an inner cavity of a catheter or endoscope. The guide wire 1 includes a core wire (wire main body) 2, which includes a core wire (wire material) having flexibility or bendability, and a coating layer 3 for coating the core wire 2.

Although not particularly limited, the total length of the guide wire 1 is preferably around 200 mm to 5000 mm.

The core wire 2 is constituted by a single continuous core wire (e.g., wire material). However, the guide wire 1 is not limited by this aspect and the core wire 2 is allowed to be a wire which is formed by joining and interlocking a plurality of core wires (wire materials) composed of the same or different materials, for example, by welding.

The core wire 2 includes a main body portion 21 positioned on the proximal side, a distal portion 22 provided on the distal side of the main body portion 21, and a taper portion 23 for interlocking the main body portion 21 and the distal portion 22.

The outer diameter of the main body portion 21 is constant approximately over the entire area in the axial line direction. Also, the transverse cross-sectional shape of the main body portion 21 is a circle. However, the shape of main body portion 21 is no limited by that shape and, for example, the main body portion 21 can include at least one taper portion and having differences in an outer diameter at respective portions in the axial line direction of the main body portion 21.

On the distal side of the main body portion 21, the taper portion 23 is provided without a level difference with respect to the main body portion 21. Regarding the taper portion 23, there is a gradual decrease in cross-sectional area from the proximal side of the core wire 2 toward the distal side thereof. Also, the transverse cross-sectional shape of the taper portion 23 is a circle. By inclusion of such a taper portion 23, the rigidity (e.g., bending rigidity, twisting rigidity) of the core wire 2 towards the distal end direction gradually decreases. As a result thereof, the guide wire 1 obtains excellent flexibility at its distal portion, whereby there is improvement in conformity to the blood vessel and safety, and concurrently it is possible to prevent bending or the like.

On the distal side of the taper portion 23, the distal portion 22 is provided. As shown in FIG. 1, the distal portion 22 is constituted by four easily-deformable portions (or deformable portions) arranged in the axial line direction of the core wire 2, which includes a first easily-deformable portion 221, a second easily-deformable portion 222, a third easily-deformable portion 223 and a fourth easily-deformable portion 224.

Each of the easily-deformable portions 221 to 224 is easily-deformable in a specific direction on a plane (that is, xy-plane) with the axial line (z-axis) of the core wire 2 being a normal line. In the xy-plane, each of the easily-deformable portions 221 to 224 deforms in one direction more easily than in other directions on this plane. The direction in which each of the easily-deformable portions 221 to 224 deforms more easily than in other directions is hereinafter referred to also as “easily-deformable direction” for the sake of convenience of explanation.

Also, the respective easily-deformable portions 221 to 224 are provided such that the easily-deformable directions thereof are different from one another.

The first easily-deformable portion 221, the second easily-deformable portion 222, the third easily-deformable portion 223 and the fourth easily-deformable portion 224 are arranged in this order (arranged in the axial line direction of the core wire 2) from the distal side of the core wire 2 toward the proximal side thereof. Among these easily-deformable portion 221 to 224, the fourth easily-deformable portion 224 positioned on the most proximal side is interlocked with the distal end of the taper portion 23. Also, the first easily-deformable portion 221 positioned on the most distal side constitutes the most distal end of the core wire 2.

The adjacent easily-deformable portions among the easily-deformable portion 221 to 224 are provided in an interlocked manner without other regions intervening. More specifically, the second easily-deformable portion 222 is provided so as to be interlocked with the first easily-deformable portion 221, the third easily-deformable portion 223 is provided so as to be interlocked with the second easily-deformable portion 222, and the fourth easily-deformable portion 224 is provided so as to be interlocked with the third easily-deformable portion 223. Thus, it is possible to shorten the total length of the distal portion 22, so that the operability of the guide wire 1 improves.

Also, the interlock portion between the first easily-deformable portion 221 and the second easily-deformable portion 222, the interlock portion between the second easily-deformable portion 222 and the third easily-deformable portion 223 and the interlock portion between the third easily-deformable portion and the fourth easily-deformable portion 224 are respectively positioned on a central axis J of the core wire 2. Thus, it becomes easy to transmit torque to the distal portion 22, and also, it is possible to reliably transmit to the most distal end the press force from the hand side, so that the operability of the guide wire 1 improves.

Subsequently, there will be an explanation regarding the constitutions of the respective easily-deformable portions 221 to 224, in which the respective easily-deformable portion 221 to 224 have similar constitutions, so that hereinafter, the constitution of the first easily-deformable portion 221 will be explained representatively, and explanation of those of the other easily-deformable portions 222 to 224 will be omitted.

As shown in FIG. 1, regarding the first easily-deformable portion 221, the plan-view shape thereof is a rectangular plate-piece shape (flat-plate shape). Also, the first easily-deformable portion 221 is provided such that the long side direction thereof coincides with the axial line direction (z-axis direction) of the core wire 2. For such a first easily-deformable portion 221, bending toward the surface direction thereof is easy compared with bending toward other directions. More specifically, the surface direction of the first easily-deformable portion 221 coincides with the easily-deformable direction thereof.

In this manner, by forming the first easily-deformable portion 221 in a plate-piece shape, it is possible to make the constitution of the first easily-deformable portion 221 simple.

Although not particularly limited, the length L (length in long-side direction) of the first easily-deformable portion 221 is preferably approximately 0.1 mm to 50.0 mm, and more preferably approximately 1.0 mm to 10.0 mm. Thus, it is possible to bend the first easily-deformable portion 221 comparatively greatly toward the easily-deformable direction while suppressing the total length of the distal portion 22.

Also, although not particularly limited, the width W (length in short-side direction) of the first easily-deformable portion 221 is preferably greater than the minimum diameter of the taper portion 23 and preferably shorter than the maximum diameter of the taper portion 23 (diameter of the main body portion 21). Specifically, the width W varies depending on the diameter of the taper portion 23, but the width is preferably approximately 0.1 mm to 1.0 mm, and more preferably around 0.5 mm to 0.9 mm. Thus, it is possible to maintain the mechanical strength of the first easily-deformable portion 221 sufficiently, and concurrently, suppress the wide width of the first easily-deformable portion 221, thereby making it possible to keep the distal portion 22 comparatively thin and to achieve improvement in the operability of the guide wire 1.

Also, although not particularly limited, the thickness T of the first easily-deformable portion 221 is preferably approximately 0.001 mm to 1.0 mm, and more preferably approximately 0.005 mm to 0.3 mm. Thus, while securing excellent curvature of the first easily-deformable portion 221 toward the easily-deformable direction, it is possible to secure mechanical strength of the first easily-deformable portion 221 sufficiently.

According to this embodiment, it is preferable for the first easily-deformable portion 221 to the fourth easily-deformable portion 224 to have mutually the same shape & size. The respective easily-deformable portions 221 to 224 having such shapes are provided such that the surface directions (that is, easily-deformable directions) of the adjacent easily-deformable portions are different from each other. By employing such an arrangement, it is possible to bend the most distal end at a proper easily-deformable portion when it goes over a lateral branch.

As shown in FIG. 2, the first easily-deformable portion 221 is provided such that the surface direction thereof (direction perpendicular to the surface) coincides with the x-axis direction. Also, regarding the second easily-deformable portion 222, the surface direction thereof inclines by approximately 45° clockwise in FIG. 2 (one circumferential direction of the core wire 2 (around the central axis J)) with respect to the surface direction of the first easily-deformable portion 221. As for the third easily-deformable portion 223, the surface direction thereof inclines by approximately 45° clockwise in FIG. 2 with respect to the surface direction of the second easily-deformable portion 222. As for the fourth easily-deformable portion 224, the surface direction thereof inclines by approximately 45° clockwise in FIG. 2 with respect to the surface direction of the third easily-deformable portion 223.

With the distal portion 22 having such a constitution, even if stress from any direction is added thereto, at least one easily-deformable portion among the first easily-deformable portion 221 to the fourth easily-deformable portion 224 is bends (deforms) in the easily-deformable direction thereof.

Specifically, when a “first stress (stress in y-axis direction, which also includes stress inclined toward z-axis direction)” shown in FIG. 2 is added to the distal portion 22, as shown in FIG. 3, mainly the first easily-deformable portion 221 easily bends toward its surface direction (easily-deformable direction). Thus, the distal portion 22 bends in accordance with the first stress.

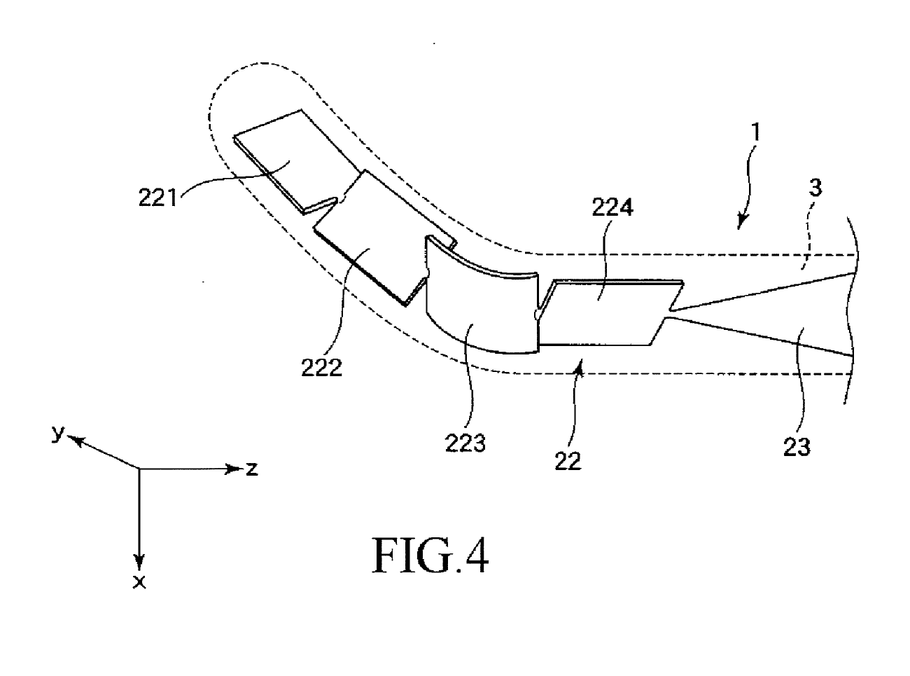

Also, when a “second stress (stress in y-axis direction, which also includes stress inclined toward z-axis direction)” shown in FIG. 2 is added to the distal portion 22, as shown in FIG. 4, mainly the third easily-deformable portion 223 easily bends toward its surface direction (easily-deformable direction). Thus, the distal portion 22 bends in accordance with the second stress.

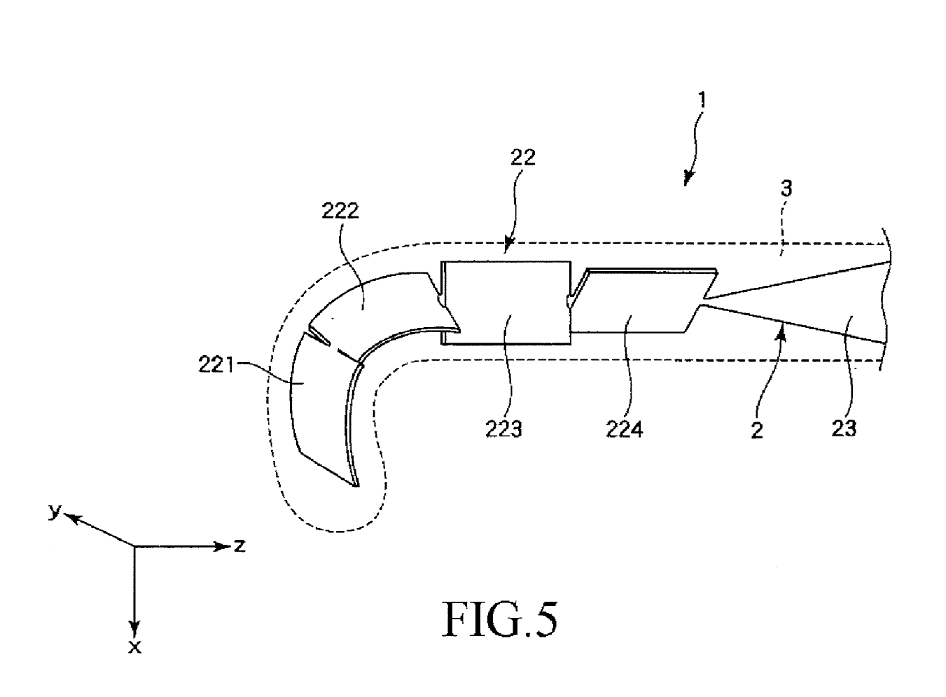

In addition, when a “third stress (stress in a direction between the surface direction of the first easily-deformable portion 221 and the surface direction of the second easily-deformable portion 222, which also includes stress inclined to z-axis direction)” shown in FIG. 2 is added to the distal portion 22, as shown in FIG. 5, the first easily-deformable portion 221 and the second easily-deformable portion 222 easily bend toward the surface directions, respectively. Thus, the distal portion 22 bends in accordance with the third stress while twisting.

According to a guide wire 1, as shown in FIG. 6A, even if the most distal end of the guide wire 1 gets into a lateral branch, at least one easily-deformable portion 221 to 224 among a plurality of easily-deformable portions 221 to 224 which are positioned at the distal portion of the guide wire 1 bends toward the easily-deformable direction thereof (see FIG. 6B) when the guide wire 1 is operated so as to advance toward the main blood vessel, whereby the most distal end of the guide wire 1 is returned to the main blood vessel and advances in the main blood vessel (see FIG. 6C). In this manner, erroneous intrusion of the guide wire 1 toward a lateral branch is prevented and/or suppressed.

In this embodiment, the surface directions (easily-deformable directions) of the respective easily-deformable portions 221 to 224 deviate continuously in one circumferential direction of the core wire 2 (clockwise in FIG. 2) in sequence from the first easily-deformable portion 221 on the most distal side toward the fourth easily-deformable portion 224 on the most proximal side. As shown in FIG. 2, when the angle formed between the surface of the first easily-deformable portion 221 and the surface of the second easily-deformable portion 222 in a clockwise direction is denoted by 81, the angle formed between the surface of the first easily-deformable portion 221 and the surface of the third easily-deformable portion 223 is denoted by 82, and the angle formed between the surface of the first easily-deformable portion 221 and the surface of the fourth easily-deformable portion 224 is denoted by θ3, the relationship θ1<θ2<θ3 is satisfied. By employing such a constitution, the operability of the guide wire 1 improves and it is possible to prevent the guide wire's erroneous intrusion into a lateral branch much more reliably.

More specifically, as mentioned above, in a case in which stress from a direction which belongs to any one of the easily-deformable directions of the respective easily-deformable portions 221 to 224 is added to the distal portion 22, the corresponding easily-deformable portion among the respective easily-deformable portions 221 to 224 bends toward the easily-deformable direction thereof. Alternatively, in a case in which stress from a direction which does not belong to any of the easily-deformable directions of the respective easily-deformable portions 221 to 224 is added to the distal portion 22, adjacent two easily-deformable portions bend toward the easily-deformable directions, respectively. In this manner, with stress added from any direction, the distal portion 22 bends in one comparatively narrow area in the axial line direction, so that it is possible to reduce the curvature radius of the bent portion. Therefore, it is possible to return the most distal end from a lateral branch to the main blood vessel easily and prevent the guide wire's 1 erroneous intrusion into a lateral branch more effectively.

Also, in this embodiment, the continuous deviations of the respective easily-deformable portions 221 to 224 are set at even intervals (e.g., intervals of 45°). Therefore, the distal portion 22 deforms approximately uniformly even with respect to stress from any direction. Therefore, the operability of the guide wire 1 improves further.

Also, in this embodiment, the easily-deformable direction of the first easily-deformable portion 221 positioned on the most distal side and the easily-deformable direction of the fourth easily-deformable portion 224 positioned on the most proximal side deviate from each other by 90° or more in the aforementioned one circumferential direction of the core wire 2. Therefore, the distal portion 22 deforms approximately uniformly even with respect to stress from any direction. Thus, the operability of the guide wire 1 improves further and it is possible to prevent its erroneous intrusion into a lateral branch more effectively.

Also, in this embodiment, the first easily-deformable portion 221 comprises the distal end of the core wire 2, so that a region nearer to the most distal end of the guide wire 1 bends easily. Therefore, even if the most distal end of the guide wire 1 gets into a lateral branch, the distal portion of the guide wire 1 bends immediately and the most distal end is returned to the main blood vessel. Therefore, according to such a constitution, it is possible to prevent erroneous intrusion of the guide wire 1 into a lateral branch more effectively.

It is possible for such a distal portion 22 (each of the easily-deformable portions 221 to 224) to be formed easily, for example, by pressing a core material, whose transverse cross-sectional shape is a circle, in a state in which it is arranged in a die. However, the forming method of the distal portion 22 is not limited by this method and, for example, it is allowed to be formed by twisting a flat-plate shaped wire material and it is also allowed to be formed by welding flat-plate shaped wire materials previously cut into predetermined size.

There is no limitation in particular for the material of the core wire 2 and, for example, there can be cited various kinds of metal materials such as iron-based alloys (alloys in which iron is the main component) such as stainless steel (for example, all kinds of SUS such as SUS304, SUS303, SUS302, SUS316, SUS316L, SUS316J1, SUS316J1L, SUS405, SUS430, SUS434, SUS444, SUS429, SUS430F, SUS302 and the like), piano wire, iron-cobalt-based alloy, carbon steel (including ultra low carbon steel, low carbon steel or the like), mild steel, hard steel, nickel steel, nickel chrome steel, nickel chrome molybdenum steel and the like; in addition, cobalt-based alloy; titanium-based alloy; nickel-based alloy; and the like. Among these, stainless steel has high strength and rigidity compared with super-elastic alloy mentioned later and therefore, it is possible to impart excellent pressability and torque transmissibility to the guide wire 1 and thus, in an embodiment stainless steel is preferable.

In addition, for the material of the core wire 2, it is possible to use an alloy (including super-elastic alloy) exhibiting pseudoelasticity and in particular, it is preferable to use a super-elastic alloy as the alloy exhibiting pseudoelasticity.

The super-elastic alloy is rich in flexibility, has restorability and will not easily get into the habit of bending, so that by constituting the core wire 2 (in particular, the distal portion thereof) by a super-elastic alloy, it is possible for the guide wire 1 to get sufficient flexibility and restorability against bending at the portion on the distal side thereof, its conformity to a blood vessel or the like, which curves & bends intricately, improves and more excellent operability can be obtained and concurrently, it will not get into the habit of bending by the restorability inherent in the core wire 2 even if the core wire 2 repeatedly undergoes curving & bending deformation, thereby making it possible to prevent reduction in operability caused by the core wire 2 getting into the habit of bending while the guide wire 1 is being used.

The pseudoelastic alloy includes any form represented by a stress-distortion curve created by pulling, and includes all alloys which greatly deform (distort) by stress and virtually return to their original shapes by removal of the stress, including those whose transformation points can be noticeably measured such as As, Af, Ms, Mf and the like or cannot be measured.

As a preferable composition of the super-elastic alloy, it is possible to cite an Ni—Ti-based alloy such as an Ni—Ti alloy containing Ni by 49 atom % to 52 atom %, a Cu—Zn alloy containing Zn by 38.5 weight % to 41.5 weight %, a Cu—Zn—X alloy containing X by 1 weight % to 10 weight % (X is at least one kind among Be, Si, Sn, Al and Ga), an Ni—Al alloy containing Al by 36 atom % to 38 atom % Al, and the like. Among these compositions, what is preferable in particular is the abovementioned Ni—Ti-based alloy. The super-elastic alloy typified by the Ni—Ti-based alloy is excellent in terms of adhesion to a coating layer 3 mentioned later.

The cobalt-based alloy has a high elasticity modulus when used as a wire and also has a suitable elastic limit. Consequently, the wire constituted by the cobalt-based alloy is excellent in torque transmissibility and thus problems such as buckling and the like are very unlikely to occur. As the cobalt-based alloy, it is allowed to use any alloy if it contains Co as a constituent element, and it is preferable to use an alloy containing Co as a main component (Co-based alloy: alloy whose Co content rate by weight ratio is the highest among the elements constituting the alloy) and more preferable to use a Co—Ni—Cr-based alloy. By using an alloy having such a composition, the abovementioned effect becomes even more remarkable. In addition, by the fact that the alloy having such a composition has a high elastic coefficient, also at the same time, can be molded by cold forming even with a high elastic limit and has a high elastic limit, it is possible to realize a reduction in diameter while sufficiently preventing the occurrence of buckling and it is possible to obtain an alloy provided with enough flexibility and rigidity to be inserted into a predetermined region.

As mentioned above, the core wire 2 is also allowed to be a wire formed by interlocking a plurality of core wires (wire materials) composed of different materials and, for example, it is possible to constitute the wire by a first core wire on the distal side and a second core wire joined to the proximal end of the first core wire. In this case, the first core wire is preferably constituted by the abovementioned super-elastic alloy and particularly preferably constituted by a Ni—Ti-based alloy, and the second core wire is preferably constituted by the abovementioned stainless steel. Then, it is allowed for the boundary portion (joined portion) between the first core wire and the second core wire to be positioned at a location more proximal than the taper portion 23, the proximal end of the taper portion 23, and a location along the taper portion 23.

On the outer circumferential surface of the core wire 2, a coating layer 3 contacts the whole or a portion of that wire. This coating layer 3 covers the whole of the core wire 2 according to the constitution shown in FIG. 1. It is possible to form the coating layer 3 for various purposes, and in particular, it is preferable to from the layer for the purpose of reducing the friction (slide resistance) of the guide wire 1 and improving the slidability thereof. Thus, the operability of the guide wire 1 improves. In particular, by covering the distal portion 22 with the coating layer 3, it is possible to form the surface of the distal portion of the guide wire 1 without a level difference.

There is no limitation in particular for the thickness of the coating layer 3 and the thickness is appropriately selected considering the formation purpose, material, forming method and the like of the coating layer 3, but usually, it is preferable for the (average) thickness thereof to be approximately 30 μm to 300 μm and more preferably, approximately 50 μm to 200 μm. When the thickness of the coating layer 3 is too small, there is a case in which the formation purpose of the coating layer 3 cannot be sufficiently realized. Also, when the thickness of the coating layer 3 is too great, there threatens to be a case in which the physical properties of the core wire 2 (guide wire 1) are influenced. In accordance with an embodiment, the coating layer 3 can be a laminated body having two or more layers.

Also, the distal end surface of the coating layer 3 partakes of roundness. Thus, at the time of insertion of the guide wire 1 into a blood vessel or the like, it is possible to more reliably prevent the distal end surface of the coating layer 3 (guide wire 1) from injuring a bile duct, an inner wall of the blood vessel or the like.

The resin constituting the coating layer 3 is not particularly limited and, for example, there can be cited polyolefins such as polyurethane, polyethylene, polypropylene, ethylene-propylene copolymer and the like, fluorine-based resins such as polytetrafluoroethylene and the like, polyesters such as polyethylene terephthalate and the like, polyvinyl chloride, polyamide, polyimide, ethylene-vinyl acetate copolymer, ethylene-ethylene acrylate copolymer, ABS resin, AS resin, butadiene-styrene copolymer, polyisoprene, polybutadiene and the like, and it is possible to use one kind, or two or more kinds of these in combination, and in particular, for the reason of superiority in flexibility and in adhesion to the core wire 2, it is preferable to use a material comparatively high in flexibility, such as polyurethane or the like.

Also, it is preferable for at least the outer surface of the distal portion of the guide wire 1 to be coated with a hydrophilic material. Thus, the hydrophilic material becomes wet and exhibits lubricity, the friction (slide resistance) of the guide wire 1 lowers and the slidability thereof improves. Therefore, the operability of the guide wire 1 improves.

As the hydrophilic material, it is possible to cite, for example, a cellulose-based polymer material, a polyethylene oxide-based polymer material, a maleic anhydride-based polymer material (for example, maleic anhydride copolymer such as methylvinylether-maleic anhydride copolymer), an acrylamide-based polymer material (for example, polyacrylamide, block copolymer of polyglycidyl methacrylate-dimethylacrylamide (PGMA-DMAA)), water-soluble nylon, polyvinylalcohol, polyvinylpyrrolidone, or the like.

In many cases, such a hydrophilic material exhibits lubricity by wetting (water absorption) and reduces the frictional resistance (slide resistance) with respect to the inner wall of an endoscope or the catheter (tube body) used together with the guide wire 1. Thus, the slidability of the guide wire 1 improves and the operability of the guide wire 1 inside the catheter becomes more excellent.

In accordance with an embodiment, the outer circumferential surface (front surface) of the core wire 2 can be subjected to a treatment (roughening treatment, chemical treatment, thermal treatment or the like) for improving adhesion to the coating layer 3.

Also, to the region positioned at the distal portion of the guide wire 1 of the coating layer 3, a contrast agent composed of metal powder (metal particles) having an X-ray contrast property can be added. There is no limitation in particular for this metal material and, for example, there can be cited tungsten and precious metals such as gold, platinum and the like, with tungsten being particularly preferable. Thus, when inserting the guide wire 1 into a target region of a body lumen such as a bile duct under X-ray fluoroscopic control, it is possible to reliably comprehend where the distal portion of the guide wire 1 is positioned in the body lumen. The average particle diameter (average diameter) of the contrast agent in the coating layer 3, is, for example, preferably approximately 0.5 μm to 4.0 μm, and more preferably approximately 1.0 μm to 1.5 μm.

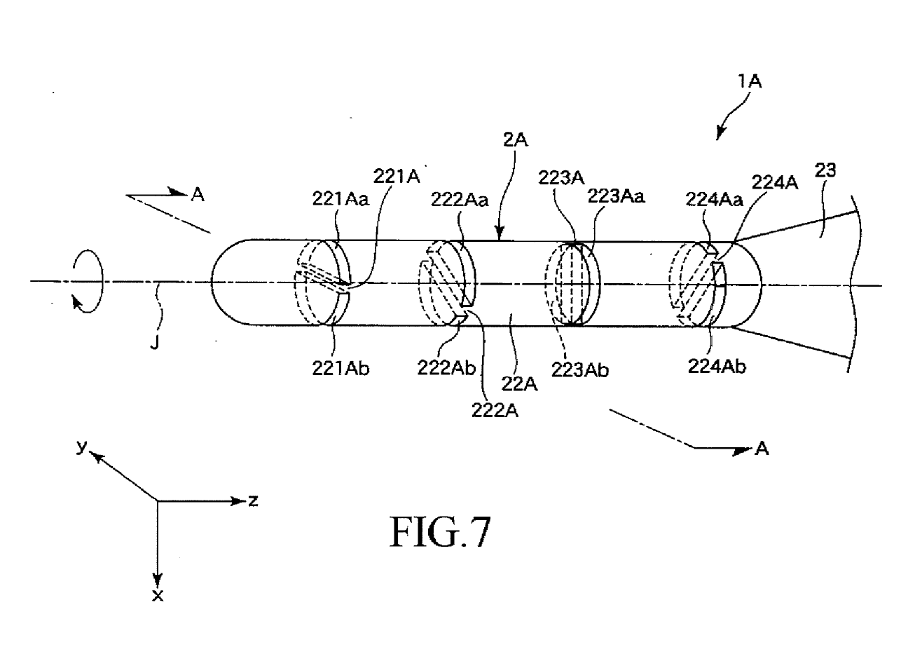

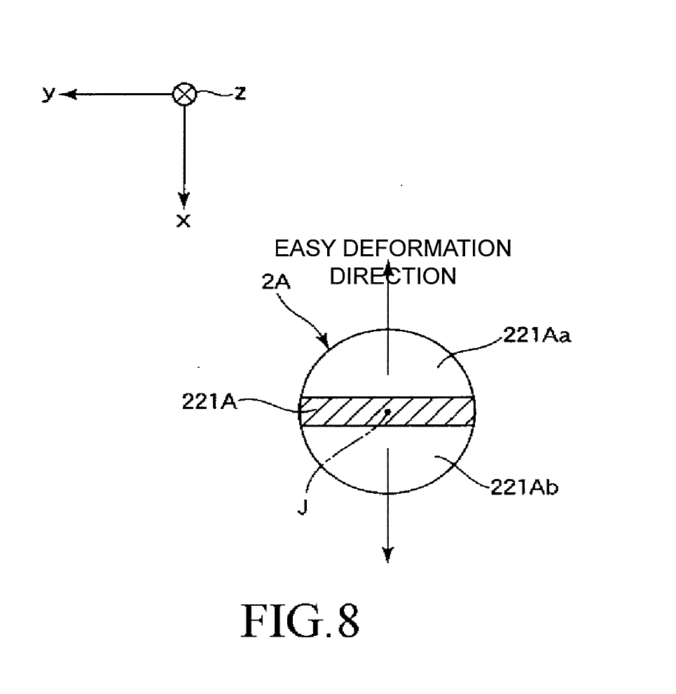

FIG. 7 is a perspective view showing a second embodiment of a guide wire disclosed by way of example. FIG. 8 is a cross-sectional view taken along the A-A line in FIG. 7. In FIGS. 7 and 8, a graphic indication of a coating layer has been omitted.

Hereinafter, there will be explained the second embodiment of the guide wire with reference to these drawings, but explanations will be made centering on the differences with respect to the abovementioned embodiment and explanations will be omitted regarding similar matters.

As shown in FIG. 7, at a distal portion 22A of a guide wire 1A, a pair of notch portions formed so as to face each other through the central axis J of a core wire 2A are plurally provided, arranged in the axial direction of the core wire 2A. Specifically, on the most distal side of the distal portion 22A, a pair of notch portions 221Aa, 221Ab is formed. On the proximal sides of the pair of notch portions 221Aa, 221Ab, there are formed a pair of notch portions 222Aa, 222Ab, which are spaced apart from those portions. On the proximal sides of the pair of notch portions 222Aa, 222Ab, there are formed a pair of notch portions 223Aa, 223Ab, which are spaced apart from those portions. On the proximal sides of the pair of notch portions 223Aa, 223Ab, there are formed a pair of notch portions 224Aa, 224Ab, which are spaced apart from those portions.

In accordance with an embodiment, the pair of notch portions 221Aa, 221Ab; the pair of notch portions 222Aa, 222Ab; the pair of notch portions 223Aa, 223Ab; and the pair of notch portions 224Aa, 224Ab are formed at even intervals respectively in the axial line direction of the core wire 2A. Although not particularly limited, the interval is, for example, preferably approximately 0.001 mm to 0.5 mm and more preferably approximately 0.05 mm to 0.2 mm. Thus, it is possible to suppress the total length of the distal portion 22A, so that the operability of the guide wire 1A improves.

In the guide wire 1A, a region where the pair of notch portions 221Aa, 221Ab of the distal portion 22A are formed constitutes a first easily-deformable portion 221A, a region where the pair of notch portions 222Aa, 222Ab are formed constitutes a second easily-deformable portion 222A, a region where the pair of notch portions 223Aa, 223Ab are formed constitutes a third easily-deformable portion 223A and a region where the pair of notch portions 224Aa, 224Ab are formed constitutes a fourth easily-deformable portion 224A.

As shown in FIG. 8, the pair of notch portions 221Aa, 221Ab are formed so as to face each other through the central axis J of the core wire 2A. Each of the notch portions 221Aa, 221Ab is formed in a groove shape, which does not reach the central axis J. Also, the bottom surface of each of the notch portions 221Aa, 221Ab is constituted by a flat surface. Also, the bottom surfaces of the respective notch portions 221Aa, 221Ab are made parallel with each other through the central axis J. Such notch portions 221Aa, 221Ab are formed to have approximately the same shapes & sizes as each other.

Although not particularly limited, the length of each of the notch portions 221Aa, 221Ab in the axial line direction of the core wire 2A is preferably approximately 0.001 mm to 5.0 mm and more preferably approximately 0.05 mm to 1.0 mm. Thus, it is possible to make the deformation amounts of the respective easily-deformable portions 221A to 224A large while suppressing the total length of the distal portion 22A.

As shown in FIG. 8, the first easily-deformable portion 221A which is constituted by forming such a pair of notch portions 221Aa, 221Ab has a long shape extending in the y-axis direction when seen with the xy-plane. Such a first easily-deformable portion 221A is easily-deformable toward the direction perpendicular to the extending direction thereof, that is, toward the x-axis direction.

With regard to the second easily-deformable portion 222A constituted by forming the pair of notch portions 222Aa, 222Ab; the third easily-deformable portion 223A constituted by forming the pair of notch portions 223Aa, 223Ab; and the fourth easily-deformable portion 224A constituted by forming the pair of notch portions 224Aa, 224Ab, constitutions similar to that of the first easily-deformable portion 221A are employed, so that explanation thereof will be omitted.

The second easily-deformable portion 222A deviates by approximately 45° in the arrow direction in FIG. 7 (in one direction around the central axis J of the core wire 2′) with respect to the first easily-deformable portion 221A. More specifically, the pair of notch portions 222Aa, 222Ab deviate by approximately 45° in the arrow direction in FIG. 7 with respect to the pair of notch portions 221Aa, 221Ab. Similarly to this, the third easily-deformable portion 223A deviates by approximately 45° in the arrow direction in FIG. 7 with respect to the second easily-deformable portion 222A and the fourth easily-deformable portion 224A deviates by approximately 45° in the arrow direction in FIG. 7 with respect to the third easily-deformable portion 223A.

In this manner, by forming the notch portions on the distal portion 22A, it is possible to form the respective easily-deformable portions 221A to 224A easily.

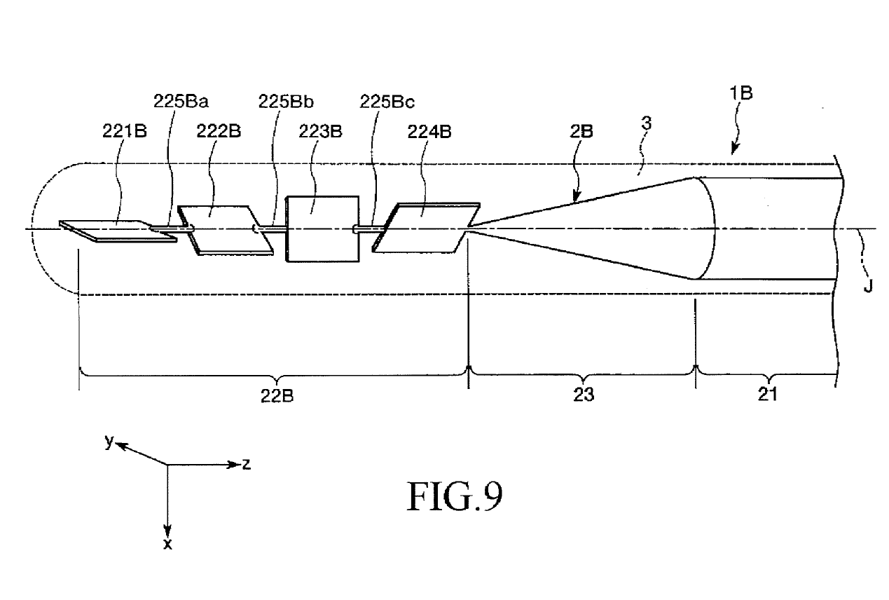

FIG. 9 is a perspective view showing a third embodiment of a guide wire disclosed by way of example.

Hereinafter, there will be explained the third embodiment of the guide wire with reference to this drawing, but explanations will be made centering around the differences with respect to the abovementioned embodiments and explanations will be omitted regarding similar matters.

As shown in FIG. 9, on the distal side of a guide wire 1B, there is provided a distal portion 22B. As shown in FIG. 9, the distal portion 22B is constituted by four easily-deformable portions, that is, a first easily-deformable portion 221B, a second easily-deformable portion 222B, a third easily-deformable portion 223B and a fourth easily-deformable portion 224B, which are arranged in the axial line direction of a core wire 2B. These four easily-deformable portions 221B to 224B have similar constitutions to those of the easily-deformable portions 221 to 224 of the abovementioned first embodiment, so that explanation thereof will be omitted.

In the guide wire 1B, the four easily-deformable portions 221B to 224B are interlocked respectively through line shaped portions 225Ba to 225Bc which are in the shape of lines. Specifically, the first easily-deformable portion 221B and the second easily-deformable portion 222B are interlocked through the line shaped portion 225Ba, the second easily-deformable portion 222B and the third easily-deformable portion 223B are interlocked through the line shaped portion 225Bb, and the third easily-deformable portion 223B and the fourth easily-deformable portion 224B are interlocked through the line shaped portion 225Bc.

The line shaped portions 225Ba to 225Bb are provided coaxially with respect to one another and are positioned on the central axis J of the core wire 2B. Thus, it becomes easy to transfer torque to the distal portion 22B and the operability of the guide wire 1B improves. Further, the flexibility of the distal portion 22B heightens, so that the safety of the guide wire 1B improves.

Although not particularly limited, the transverse cross-sectional shape of the line shaped portions 225Ba to 225Bb is preferably a circle. Thus, it is possible to deform the line shaped portions 225Ba to 225Bc equally toward any directions in the diameter directions thereof, so that it is possible to improve the operability of the guide wire 1B.

Also, in a case in which the transverse cross-sectional shape of the line shaped portions 225Ba to 225Bb is a circle, the diameter thereof is not particularly limited but preferably approximately 0.001 mm to 0.90 mm and more preferably approximately 0.01 mm to 0.5 mm. By setting the diameters of the line shaped portions 225Ba to 225Bb within the abovementioned ranges, it is possible to strike a balance between mechanical strength and easily-deformable property of the distal portion 22B.

Also, although not particularly limited, the length of each of the line shaped portions 225Ba to 225Bb is, for example, preferably approximately 0.01 mm to 5.0 mm and more preferably approximately 0.1 mm to 1.0 mm. By setting the length of each of the line shaped portions 225Ba to 225Bb within the abovementioned ranges, it is possible to suppress the total length of the distal portion 22B and concurrently, to improve the operability of the guide wire 1B.

In the guide wire 1B having such a constitution, when stress is added to the distal portion 22B, there is created a situation in which at least one easily-deformable portion among the four easily-deformable portions 221B to 224B deforms toward the easily-deformable direction and concurrently, at least one line shaped portion among the three line shaped portions 225Ba to 225Bc deforms.

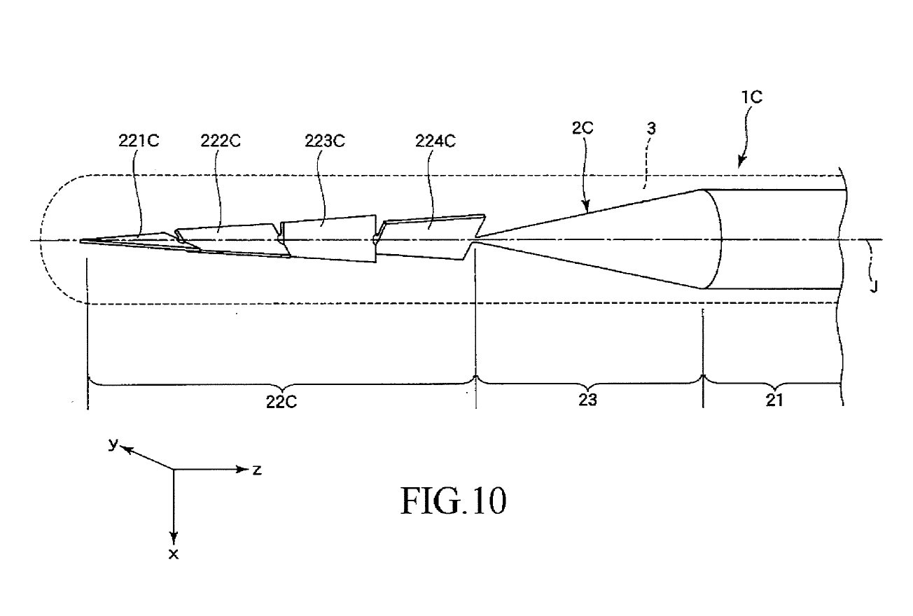

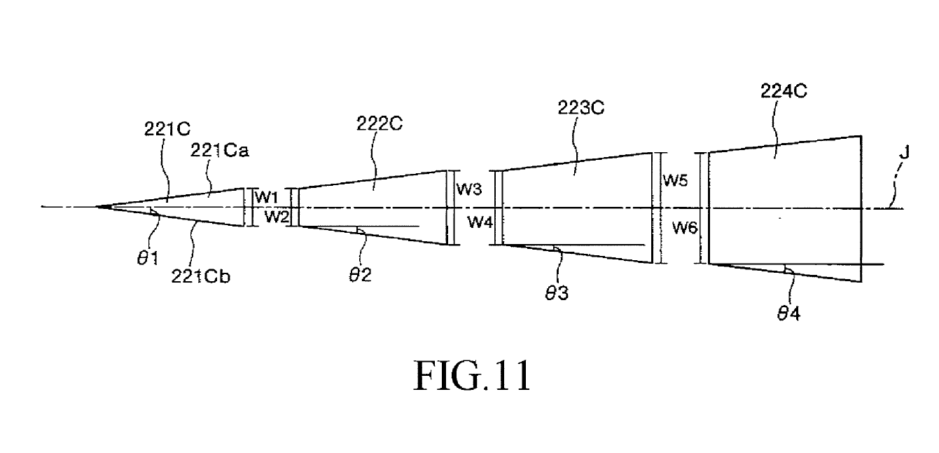

FIG. 10 is a perspective view showing a fourth embodiment of a guide wire, by way of example. FIG. 11 is an exploded view of the guide wire as shown in FIG. 10.

Hereinafter, there will be explained the fourth embodiment of the guide wire with reference to the drawings, but explanations will be made centering around the differences with respect to the abovementioned embodiments and explanations will be omitted regarding similar matters.

As shown in FIG. 10, at a distal portion 22C of a guide wire 1C, there are formed a first easily-deformable portion 221C, a second easily-deformable portion 222C, a third easily-deformable portion 223C, and a fourth easily-deformable portion 224C in sequence from the distal side.

FIG. 11 is a drawing showing an aspect in which four easily-deformable portions 221C to 224C are arranged on the same plane. As shown in FIG. 11, the easily-deformable portion (first easily-deformable portion) 221C positioned on the most distal side has a triangular plan-view shape, the other three easily-deformable portions (second easily-deformable portion, third easily-deformable portion and fourth easily-deformable portion) 222C to 224C have trapezoidal plan-view shapes respectively. Also, each of the easily-deformable portions 221C to 224C has a shape which is axially symmetrical with respect to the central axis J of the core wire 2C when seen with the plan view.

Each of the easily-deformable portions 221C to 224C has such a shape that its width (distance between the sides which face each other through the central axis J of the core wire 2C; for example, distance between the sides 221Ca, 221Cb of the first easily-deformable portion 221C) decreases gradually from the proximal side toward the distal side.

Also, regarding one pair of easily-deformable portions adjacent to each other, the width of the easily-deformable portion, which is positioned on the distal side, on the proximal side and the width of the easily-deformable portion, which is positioned on the proximal side, on the distal side are made equal to each other. For example, the width W1 of the first easily-deformable portion 221C on the proximal side and the width W2 of the second easily-deformable portion 222C on the distal side are made equal, the width W3 of the second easily-deformable portion 222C on the proximal side and the width W4 of the third easily-deformable portion 223C on the distal side are made equal, and the width W5 of the third easily-deformable portion 223C on the proximal side and the width W6 of the fourth easily-deformable portion 224C on the distal side are made equal. By employing such a constitution, the rigidity of the guide wire 1C is reduced smoothly, the operability of the guide wire 1C is improved and concurrently, safety can be improved as the distal end becomes flexible.

Also, the inclined angles θ1 to θ4 of the sides, which face one another through the central axis J of the easily-deformable portions 221C to 224C, with respect to the central axis J are made equal to one another. Although not particularly limited, for example, each of the inclined angles θ1 to θ4 is preferably approximately 5 degrees to 80 degrees and more preferably approximately 10 degrees to 45 degrees.

As described above, the guide wires as disclosed are explained regarding the embodiments shown in the drawings, but the present invention is not limited by these configurations and it is possible for the respective portions constituting the guide wire to be replaced by arbitrary constructions which can exhibit similar functions. Also, any arbitrary constituent element is allowed to be added.

Also, it is allowed for the guide wire can be a wire formed by combining two or more constitutions (characteristics) within the respective embodiments.

The use application of the guide wires disclosed by way of example, are not limited by the case in which the guide wire is used in the abovementioned trans-endoscope surgical procedure and it is possible for the guide wire to be used, for example, in an operation at the time of treating CTO (Chronic Total Occlusion), in blood vessel contrast, in PTCA and the like.

According to an aspect, if a guide wire is operated so as to advance in a main blood vessel even after the most distal end of the guide wire gets into a lateral branch, at least one easily-deformable portion among a plurality of easily-deformable portions which are positioned at the distal portion of the guide wire bends easily toward the easily-deformable direction thereof, and thus, the most distal end of the guide wire is returned to the main blood vessel and advances in the main blood vessel, so that it is possible to prevent or suppress erroneous intrusion of the guide wire into the lateral branch. Therefore, the guide wires as disclosed by way of example have industrial applicability.

The detailed description above describes a guide wire and a manner of forming the guide wire. The invention is not limited, however, to the precise embodiments and variations described above and illustrated in the drawing figures. Various changes, modifications and equivalents could be effected by one skilled in the art without departing from the spirit and scope of the invention as defined in the appended claims. It is expressly intended that all such changes, modifications and equivalents which fall within the scope of the claims are embraced by the claims.

What is claimed is:

1. A guide wire comprising:
an elongated core wire having a main body portion and a distal portion positioned on a distal side of the main body portion;
the distal portion having a plurality of adjacent plate-shaped members, each of the plurality of adjacent plate-shaped members having a surface direction on a plane of an axial line of the elongated core wire; and
wherein the surface directions of the adjacent plate-shaped members among the plurality of adjacent plate-shaped members are different from each other.

2. The guide wire according to claim 1, further comprising a tapered portion positioned between the main body portion and the distal portion, which interlocks the main body portion to the distal portion of the core wire.

3. The guide wire according to claim 2, wherein the tapered portion decreases in cross-sectional area from a proximal side to a distal side thereof.

4. The guide wire according to claim 1, wherein the plurality of plate-shaped members is comprised of four rectangular plate-shaped members, and wherein each of the adjacent plate-shaped members is at an angle of 45 degrees to one another.

5. The guide wire according to claim 1, wherein each of the plurality of plate-shaped members are separated by a line-shaped member.

6. The guide wire according to claim 1, wherein the plate-shaped members deviate at even intervals in sequence from the plate-shaped member positioned on a distal side of the core wire toward the plate-shaped member positioned on the proximal side thereof.

7. The guide wire according to claim 1, wherein the direction in which the plate-shaped members on a distal most side of the core wire among the plurality of plate-shaped members and the direction in which the plate-shaped member positioned on a most proximal side thereof deviate from each other by 45 degrees or more in a circumferential direction.

8. The guide wire according to claim 1, wherein a distal most side plate-shaped member has a triangular plan-view shape, and each of a remaining plate-shaped members of the plurality of plate-shaped members has a trapezoidal plan-view shape.

9. A guide wire comprising:
an elongated core wire having a main body portion and a distal portion positioned on a distal side of the main body portion;
the distal portion having a plurality of notch portions, each of the plurality of notch portions formed in pairs, which face each other through a central axis of the core wire; and
wherein the plurality of notch portions are located at even intervals in an axial direction of the core wire and wherein each adjacent notch portion among the plurality of notch portions deviate from each other in a circumferential direction of the core wire.

10. The guide wire according to claim 9, wherein a direction of the plurality of notch portions deviate from each other by 45 degrees or more in a circumferential direction.

11. The guide wire according to claim 9, wherein the plurality of notch portions comprise at least four notch portions.

12. A guide wire comprising:
a core wire formed from a flexible metal material;
a distal portion of the core wire, which is arranged in an axial line direction of the core wire and includes a plurality of deformable portions, each of the plurality of deformable portions having a surface direction on a plane with the axial line of the core wire, and
wherein the surface directions of adjacent deformable portions among the respective deformable portions are different from each other.

13. The guide wire according to claim 12, wherein each of the deformable portions forms a rotationally asymmetric shape with respect to the axial line of the core wire.

14. The guide wire according to claim 12, wherein each of the deformable portions forms a plate-piece shape.

15. The guide wire according to claim 14, wherein the adjacent deformable portions among the respective deformable portions are provided in an interlocked manner.

16. The guide wire according to claim 12, wherein the distal portion of the core wire includes a plurality of notch portions which are arranged in the axial direction of the core wire, the adjacent notch portions among the respective notch portions are provided so as to deviate from each other in a circumferential direction of the core wire, and regions of the core wire, in which the notch portions are formed, constitute the deformable portions.

17. The guide wire according to claim 16, wherein each notch portion is formed in pairs so as to face each other through a central axis of the core wire.

18. The guide wire according to claim 12, wherein the directions in which the respective deformable portions deviate continuously in one circumferential direction of the core wire in sequence from the deformable portion positioned on a distal side of the core wire toward the deformable portion positioned on a proximal side thereof.

19. The guide wire according to claim 18, wherein the directions in which the respective deformable portions deviate at even intervals in sequence from the deformable portion positioned on the distal side of the core wire toward the deformable portion positioned on the proximal side thereof.

20. The guide wire according to claim 18, wherein the direction in which the deformable portion positioned on a most distal side of the core wire among the plurality of deformable portions and the direction in which the deformable portion positioned on a most proximal side thereof deviate from each other by 90° or more.
